(19) United States
(12) Patent Application Publication
(10) Pub. No.: US 2011/0022115 A1
(43) Pub. Date: Jan. 27, 2011

(54) TOPICAL ANESTHESIA INDUCING APPARATUS FOR INDUCING TOPICAL ANESTHESIA AND TOPICAL ANESTHESIA INDUCING APPLICATORS AND TOPICAL ANESTHESIA INDUCING ELECTRODES THEREFORE

(76) Inventors: Michael Salzhauer, Bay Harbor, FL (US); Victor M. Gamez, Fort Lauderdale, FL (US)

Correspondence Address: ROBERT M. SCHWARTZ, P.A., P.O. BOX 221470, HOLLYWOOD, FL, 33022, US

(21) Appl. No.: 12/841,577

(22) Filed: Jul. 22, 2010

Related U.S. Application Data
(60) Provisional application No. 61/227,461, filed on Jul. 22, 2009.

(51) Int. Cl. A61N 1/04

(52) U.S. Cl. 607/46

(57) ABSTRACT

Body worn Topical Anesthesia Inducing (TAI) apparatus for inducing topical anesthesia at a desired treatment site and TAI electrodes therefor. Body worn TAI apparatus includes a body worn TAI device including a power source, a TENS generation unit for generating a TENS output signal, a control unit with a user interface for controlling operation of the TAI apparatus and at least one TAI applicator with a TAI electrode for administering the TENS output signal at the desired treatment site for inducing topical anesthesia. Body worn TAI device configurations include a hand worn TAI device, a wrist worn TAI device, and a belt worn TAI device. TAI applicators can also administer vibration stimulus and/or cold application at the desired treatment site for increased topical anesthesia.

### INDEX TO RELATED APPLICATIONS

This application claims benefit of U.S. Provisional Application No. 61/227,461 filed Jul. 22, 2009 the disclosure of which is incorporated herein by reference in its entirety.

### FIELD OF THE INVENTION

The invention relates to Topical Anesthesia Inducing (TAI) apparatus for inducing topical anesthesia for chronic and acute pain management.

### BACKGROUND OF THE INVENTION

Known methods for inducing topical analgesia for chronic and acute pain management include inter alia Transcutaneous Electrical Nerve Stimulation (TENS), mechanical conditioning and cold application.

TENS apparatus include a TENS generation unit for generating a TENS output signal, TENS electrodes coupled to the pulse generator and adapted to be placed in contact at a desired treatment site. The TENS output signal can be in the form of a pulsed DC signal, an AC signal, a pulsed DC signal followed by an AC signal, and the like. The TENS electrodes can be single channel or dual channel to combine active electrodes and return electrodes on a single electrode.

Mechanical conditioning is commonly applied by manually stretching and/or tapping an injection site before and after an injection procedure.

Cold application is commonly applied by means of ice packs and the like for inducing topical anesthesia.

Representative prior art includes inter alia the following references:

U.S. Pat. No. 4,233,986 to Tannenbaum illustrates and describes TENS apparatus including a pulse width generator for modulating the width of the pulses applied by TENs electrodes to a patient's skin.

U.S. Pat. No. 5,366,489 to Burgio et al. illustrates and describes TENS electrodes and connectors including a dual channel TENS electrode that is used in combination with an elongated applicator having a bifurcated end in order to facilitate guiding a syringe needle toward a desired injection site.

US Patent Application Publication No. US 2006/0142816 A1 to Fruitman et al. illustrates and describes a TENS electrode pad that is capable of simultaneously of delivering hot or cold thermal therapy for the relief of pain.

US Patent Application Publication No. US 2008/0188779 A1 to Vallero illustrates and describes an electrode with one or more conductive surfaces and one or more access windows allowing needles, lancets and other similar objects to pass through or near the electrode or near the electrode and into the underlying skin layer. The electrode adheres to skin and transmits mild electrical and vibration stimulation to mask pain signals caused when sharp objects penetrate the skin The stimulation is delivered by an attachable electrical unit designed to generate both the TENS and vibration stimuli while connected to the electrode.

US Patent Application Publication No. US 2008/0086159 A1 to Zweifler illustrates and describes apparatus and method for facilitating painless or near-painless injections. The apparatus includes a guide that provides vibration around an injection site and a reservoir to hold a bolus of anesthesia gel through the injection needles passes. The vibrational energy of the guide is also transferred to the needle itself, further reducing any pain associated with an injection.

Other prior art references include inter alia U.S. Pat. No. 4,676,257 to Halpern entitled Dental Anesthesia Apparatus, U.S. Pat. No. 4,924,880 to O'Neill et al. entitled Dental Anesthesia Apparatus, and the like.

### SUMMARY OF THE INVENTION

The present invention is directed towards body worn Topical Anesthesia Inducing (TAI) apparatus including a body worn TAI device and one or more TAI applicators for inducing topical anesthesia at a desired treatment site. The body worn TAI device includes a power source, a TENS generation unit for generating a TENS output signal and a controller including a user interface for controlling the TAI apparatus. The TAI applicators each include a TAI electrode for administering the TENS output signal and can also optionally administer vibration stimulus and/or cold application at the desired treatment site for increased topical anesthesia.

Body worn TAI device configurations include inter alia a hand worn TAI device, a wrist worn TAI device, a waist worn TAI device, and the like. Hand worn TAI devices preferably include a pair of digit worn TAI applicators including a rigidly mounted thumb collar and a flexible index finger cuff for inducing topical anesthesia at a desired treatment site. Hand worn TAI devices optionally include a vibration unit for vibrating the rigidly mounted thumb worn TAI applicator. Digit worn TAI applicators can optionally include a thermoelectric module for cold application at a desired treatment site.

Wrist and waist worn TAI devices include a wire connection to at least one discrete TAI applicator which may be digit worn or intended to be manually pressed against a desired treatment site or adhered thereto. Discrete TAI applicators can optionally include a built-in vibration unit for administering vibration stimulus at a desired treatment site. Discrete TAI applicators can optionally include a thermoelectric module for cold application at a desired treatment site. Wrist and waist worn TAI devices can include control units for controlling such vibration units and thermoelectric modules. Alternatively, the vibration units and the thermoelectric modules can include their respective control units.

TAI electrodes can be commercially available TENS electrodes, for example, from medical supplies vendors such as Covidien (www.covidien.com). TAI electrodes may be stored and maintained at a low temperature in a dedicated chilling chamber or a conventional freezer unit. Their chilling effect at a desired treatment site can be prolonged by the provision of a chilling mechanism in the form of a gel or liquid refrigerant filled pouch similar to reusable ice pack products. Alternatively, TAI electrodes may be provided with active chilling mechanisms for inducing topical chilling effect. One such chilling mechanism includes encapsulated separated chemical agents for inducing endothermic reaction on breaking their encapsulation barriers. Another suitable chilling mechanism includes a thermoelectric module.

The present invention affords convenient ergonomic medical devices for chronic and acute pain relief management suitable for a wide range of clinical purposes. For example, clinical practitioners may use the TAI apparatus for injection procedures, and the like. Home users may use the TAI apparatus for self injection procedures, for example, by way of auto injectors, and the like. Other potential uses include dermatologic procedures for example involving laser use. Tattooing and body piercing are other practice areas in which this invention may be employed for pain reduction.

### BRIEF DESCRIPTION OF DRAWINGS

In order to understand the invention and to see how it can be carried out in practice, preferred embodiments will now be described, by way of non-limiting examples only, with reference to the accompanying drawings in which similar parts are likewise numbered, and in which:

FIG. 1 is a combined pictorial representation and block diagram of TAI apparatus including a hand worn TAI device, a rigidly mounted thumb worn TAI applicator and a flexible index finger worn TAI applicator;

FIG. 2 is a pictorial representation showing the use of FIG. 1's TAI apparatus during a standard injection procedure;

FIG. 3 is an exploded cross section view of a basic design of a TAI electrode along line A-A in FIG. 1;

FIG. 4 is an exploded cross section view of a TAI electrode including a refrigerant filled pouch along line A-A in FIG. 1;

FIG. 5 is an exploded cross section view of a TAI electrode for enabling an endothermic reaction along line A-A in FIG. 1;

FIG. 6 is an exploded cross section view of a TAI electrode with a thermoelectric module along line A-A in FIG. 1;

FIG. 7 is a pictorial representation of TAI apparatus including a wrist worn TAI device, a discrete thumb worn TAI applicator and a discrete index finger worn TAI applicator; and

FIG. 8 is a pictorial representation of TAI apparatus including a waist worn TAI device and a discrete TAI applicator.

### DETAILED DESCRIPTION OF PREFERRED EMBODIMENTS OF THE INVENTION

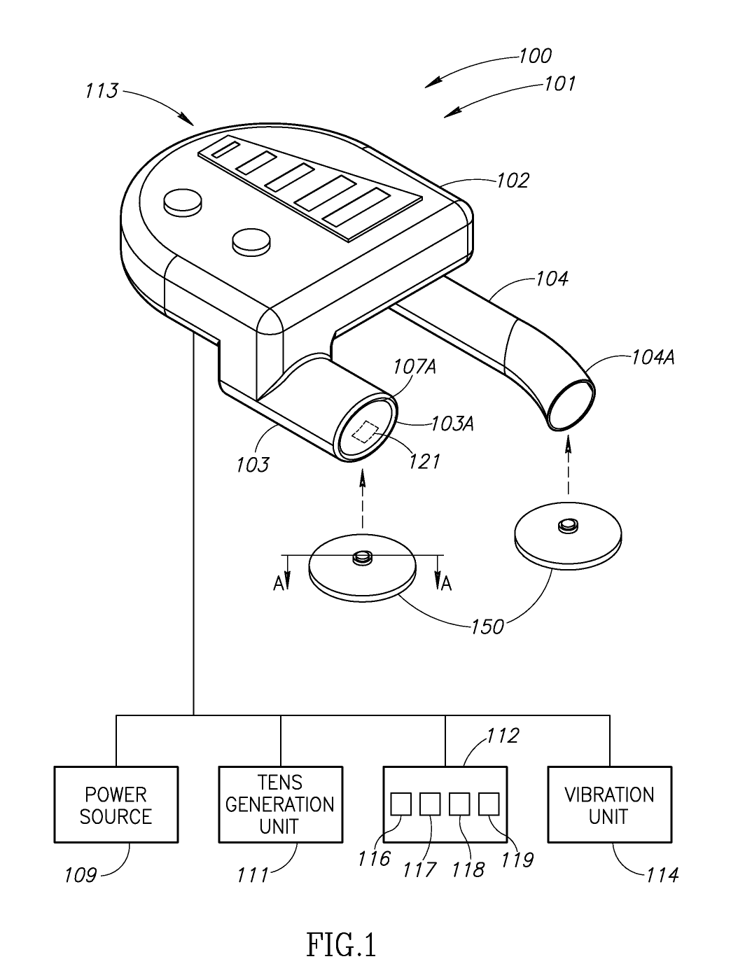

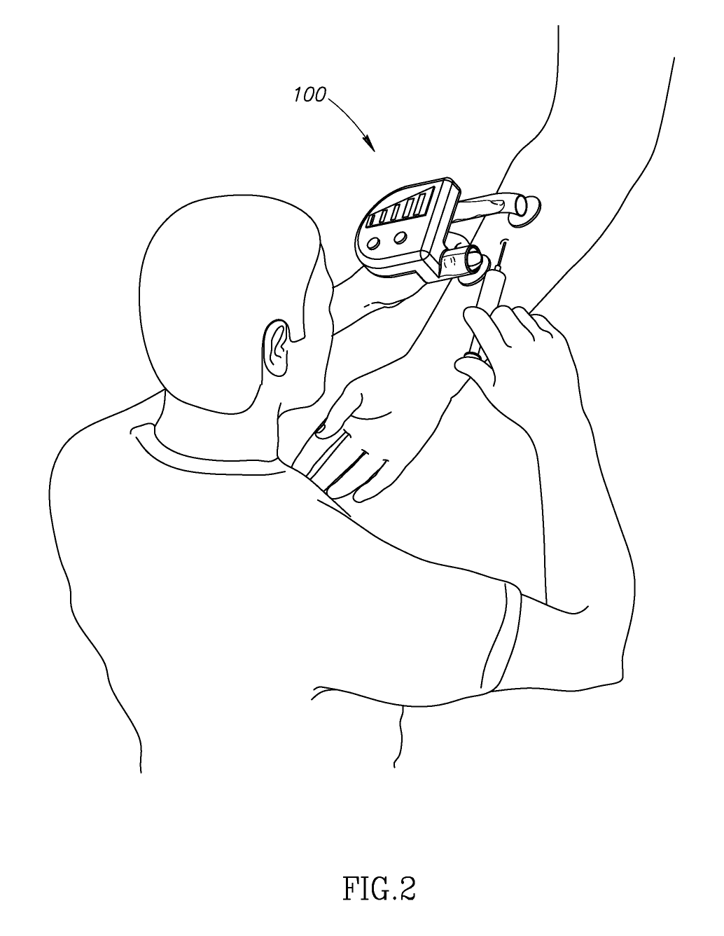

FIGS. 1 and 2 show body worn TAI apparatus 100 for inducing topical anesthesia. The body worn TAI apparatus 100 includes a hand worn TAI device 101 having a housing 102 configured to be overlying on the back of a person's hand, a thumb worn TAI applicator 103 and an index finger worn TAI applicator 104. The TAI applicators 103 and 104 constitute digit worn TAI applicators and have respective distal ends 103A and 104A each with a plug-in socket 106 for snap fit insertion of a TAI electrode 150 at an ergonomically correct angle for convenient intimate electrode contact at a desired treatment site. Suitable snap fit connections include, for example, a standard ECG connector, and the like.

The thumb worn TAI applicator 103 is preferably constituted by a rigidly mounted thumb collar 107 for sliding insertion of a person's thumb. The index finger worn TAI applicator 104 is preferably constituted by a flexible index finger cuff 108 for the sliding insertion of a person's index finger and for allowing embedding of a multitude of wires along its length and free index finger movement. The index finger cuff 108 is preferably made of flexible material such as elastomers, for example, silicone, layered elastic fabrics, and the like. The flexible index finger cuff 108 can be fixedly attached to the housing 102 or alternatively detachably connected thereto.

A basic version of the TAI device 101 includes a power source 109, a TENS generation unit 111 for issuing a TENS output signal, and a controller 112 with a user interface 113 for controlling the TAI apparatus 100. The power source 109 is preferably in the form of a commercially available battery. The housing 102 may also include a vibration unit 114 for selectively vibrating each of housing 102, the rigidly mounted thumb collar 107, individually or simultaneously which in turn will vibrate distal TAI electrode 150. Suitable vibration units 114 include, for example, vibration units deployed in cellular phones, pagers, and the like. The vibrator motor model No. 307-001 offered by Precision Microdrives (www.precisionmicrodives.com) is an example of a suitable product that may be used to this end.

The controller 112 includes an ON/OFF switch 116 and a TENS control unit 117 for controlling the TENS generation unit 111 in terms of pulse strength, pulse frequency, and the like. The user interface 113 can include visual and/or audible indicators. The controller 112 also includes a vibration control unit 118 for selectively controlling all functions of the vibration unit 114 in terms of vibration intensity, vibration frequency, duration, and the like.

The controller 112 can also include a thermoelectric module control unit 119 for controlling a thermoelectric module 121 at the rigid mounted thumb collar's distal end 107A or alternatively integrated within a TAI electrode 150D (see FIG. 6). The thermoelectric module control unit 119 controls the thermoelectric module 121 in terms of electrical supply, desired cold temperature, and the like.

The flexible index finger worn cuff 108 can be optionally provided with embedded movement sensitive components sensitive to index finger movements for input control of the controller 112. Suitable movement sensitive components include accelerometers, and the like, for converting specific index finger movements to input signals.

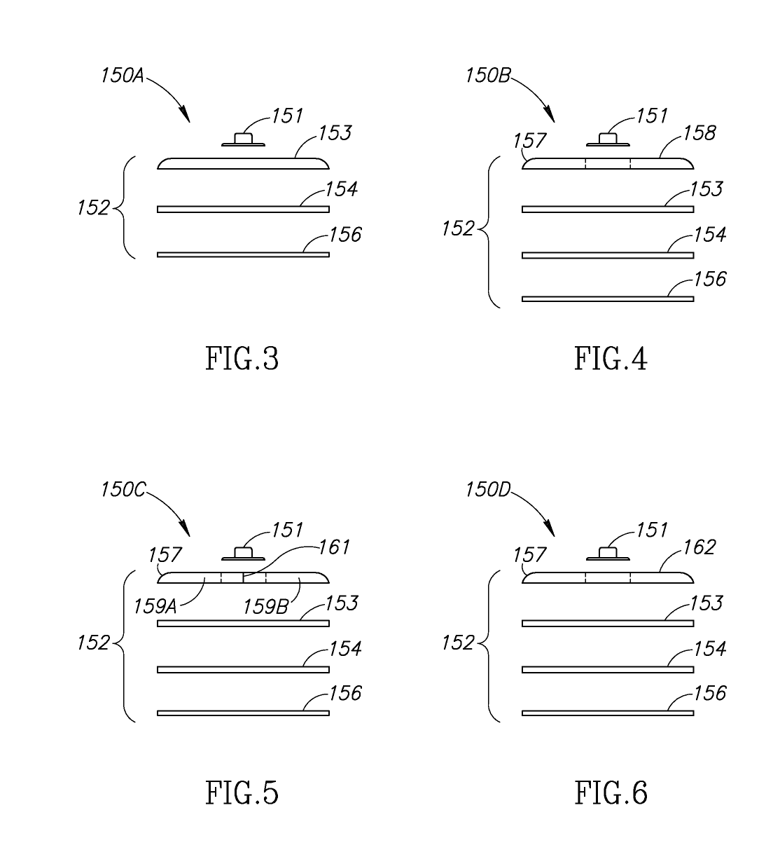

FIGS. 3 to 6 show exploded cross sections of four implementations of TAI electrodes 150 suitable for use in the TAI apparatus 100. FIG. 3 shows a TAI electrode 150A having a snap connector 151 and a layered construction 152 including a backing material 153, an electrode conductor 154 and a conductive hydrogel 156. FIG. 4 shows a TAI electrode 150B similar to the TAI electrode 150A and additionally including a chilling mechanism 157 in the form of a gel or liquid refrigerant filled pouch 158 interposed between the snap connector 151 and the backing material 153. The gel or liquid refrigerant pouch 158 is similar to reusable ice pack products. FIG. 5 shows a TAI electrode 150C similar to the TAI electrode 150B except that its chilling mechanism 157 is in the form of encapsulated chemical reagents 159A and 159B separated by an encapsulation barrier 161 for inducing an endothermic reaction on breaking same. FIG. 6 shows a TAI electrode 150D similar to the TAI electrode 150B except its chilling mechanism 157 is in the form of a thermoelectric module 162.

The use of the TAI apparatus 100 with TAI electrodes 150 is now described:

A practitioner mounts the TAI device 101 on his left hand, as seen in FIG. 2, with the housing 102 overlying the back of his hand. The left thumb is inserted into the thumb collar 107 and the left index finger is inserted into the index finger cuff 108. The practitioner snaps TAI electrodes 150 into place at the thumb collar 107's distal end and the index finger cuff 108's distal end. In the case of TAI electrodes 150A and 150B, the TAI electrodes are pre-chilled in a refrigeration unit kept preferably at about 0° Fahrenheit. In the case of TAI electrodes 150C, the practitioner breaks the encapsulation barrier 161 to induce an endothermic reaction to chill same before snapping them into place.

The practitioner sets the TENS control unit 117 and the vibration control unit 118 to correspondingly operate the TENS generation unit 111 and the vibration unit 114. The practitioner may also set a thermoelectric module control unit 119 to operate the thermoelectric module 121, if available, or TAI electrodes 150D. The practitioner intimately contacts the TAI electrodes 150 slightly apart at a desired treatment site to induce topical anesthesia. The practitioner may adjust settings as required. The TAI apparatus 100 induces topical anesthesia at the desired treatment site thereby enabling the practitioner to perform a required clinical procedure, for example, administering an injection in a painless manner.

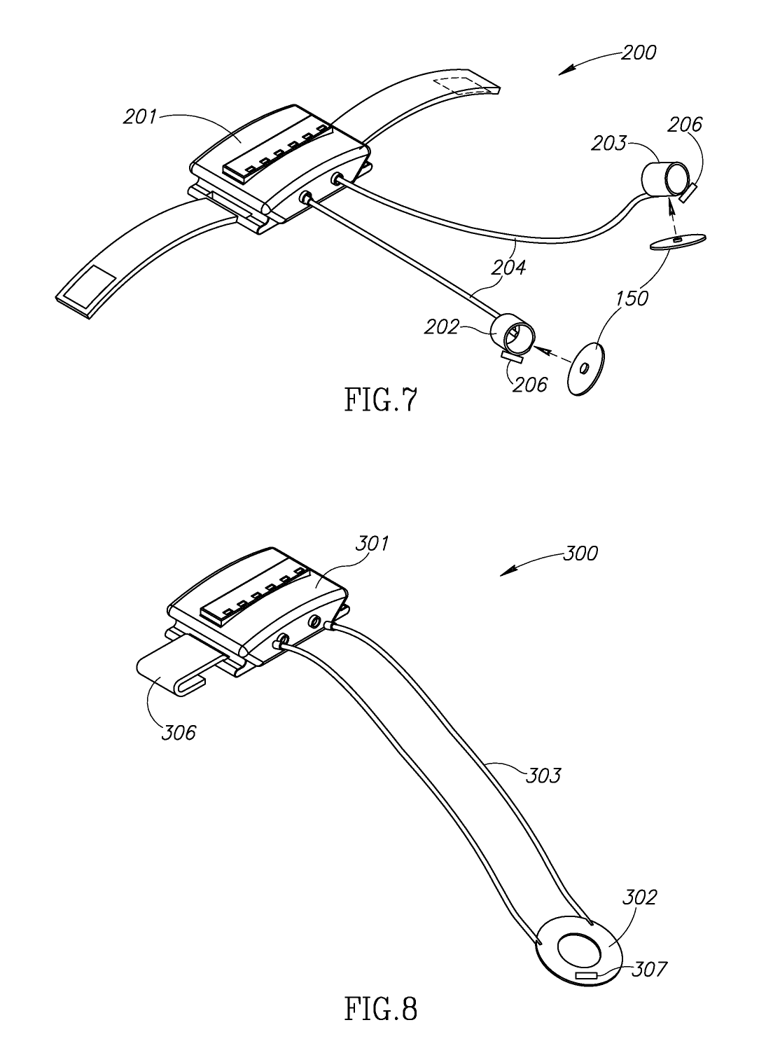

FIG. 7 shows TAI apparatus 200 similar in construction and operation as the TAI apparatus 100. The TAI apparatus 200 includes a wrist worn TAI device 201, a discrete thimble like thumb worn TAI applicator 202, a discrete thimble like index finger worn TAI applicator 203 and a wire connection 204 for connecting the TAI device 201 to the TAI applicators 202 and 203. The TAI device 201 includes the same components as the basic version of the TAI device 101. The TAI device 201's controller 112 may also be provided with the vibration control unit 118 for controlling vibration units 206 at one or both of the TAI applicators 202 and 203 for administering vibration stimulus at a desired treatment site. The TAI device 201's controller 112 may also be provided with the thermoelectric module control unit 119 for controlling thermoelectric modules 121 at one or both of the TAI applicators 202 and 203 or TAI electrodes 150D for cold application at a desired treatment site.

FIG. 8 shows TAI apparatus 300 similar in construction and operation as the TAI apparatus 100. The TAI apparatus 300 includes a waist worn TAI device 301, a discrete non digit worn TAI applicator 302, a wire connection 303 connecting the TAI device 301 to the discrete TAI applicator 302, and a belt clip 306 for clipping the TAI apparatus 300 onto a person's belt. The TAI device 301 includes the same components as the basic version of the TAI device 101. The TAI applicator 302 includes an annular TAI electrode 304 for intimate electrode contact at a desired treatment site. A person can apply pressure to the TAI electrode 304 for such intimate electrode contact. Alternatively, the TAI electrode 304 can be provided with an adhesive layer protected by a manually removable peel off layer. The TAI electrode 304 may also be optionally provided with a vibration unit 307.

The wrist worn TAI device 201 can be used with the discrete non digit mounted TAI applicator 302 and conversely the waist worn TAI device 301 can be used with the digit worn TAI applicators 202 and 203.

While the invention has been described with respect to a limited number of embodiments, it will be appreciated that many variations, modifications, and other applications of the invention can be made within the scope of the appended claims.

1. Topical Anesthesia Inducing (TAI) apparatus for inducing topical anesthesia at a desired treatment site, the TAI apparatus comprising:
(a) a body worn TAI device including a power source, a TENS generation unit for generating a TENS output signal and a controller having a user interface for controlling operation of the TAI apparatus; and
(b) at least one TAI applicator including a TAI electrode for intimate electrode contact at the desired treatment site and in electrical connection with said TENS generation unit for administering said TENS output signal at the desired treatment site for inducing topical anesthesia.

2. The TAI apparatus according to claim 1 wherein said TAI device is configured to overlie the back of a person's hand and includes a thumb worn TAI applicator and an index finger worn TAI applicator.

3. The TAI apparatus according to claim 2 wherein said TAI device includes a rigidly mounted thumb worn TAI applicator and a flexible index finger worn TAI applicator.

4. The TAI apparatus according to claim 3 wherein said TAI device further includes a vibration unit for vibrating said rigidly mounted thumb worn TAI applicator for administering vibration stimulus at the desired treatment site.

5. The TAI apparatus according to claim 2 wherein said flexible index finger worn TAI applicator includes a movement sensor sensitive to movement of the person's index finger for providing control instructions for controlling said control unit.

6. The TAI apparatus according to claim 1 wherein said TAI device includes a wire connection to at least one discrete TAI applicator.

7. The TAI apparatus according to claim 6 wherein said at least one discrete TAI applicator is a digit worn TAI applicator.

8. The TAI apparatus according to claim 6 wherein said at least one discrete TAI applicator is a non-digit worn TAI applicator.

9. The TAI apparatus according to claim 6 wherein said TAI device is configured to be attached to a person's wrist.

10. The TAI apparatus according to claim 6 wherein said TAI device is configured to be worn on a person's waist.

11. The TAI apparatus according to claim 1 wherein said TAI applicator includes a vibration unit for administering vibration stimulus at the desired treatment site.

12. The TAI apparatus according to claim 1 wherein said TAI applicator includes a thermoelectric module for chilling the desired treatment site.

13. The TAI apparatus according to claim 1 wherein said TAI apparatus includes a chilling mechanism for chilling the desired treatment site.

14. The TAI apparatus according to claim 13 wherein said chilling mechanism is constituted by a refrigerant filled pouch.

15. The TAI apparatus according to claim 13 wherein said chilling mechanism includes an encapsulation barrier separating chemical reagents for enabling an endothermic chemical reaction on breaking said encapsulation barrier.

16. The TAI apparatus according to claim 13 wherein said chilling mechanism includes a thermoelectric device.

17. A TAI applicator for use with the TAI apparatus according to claim 1.

18. A TAI electrode for use with TAI apparatus according to claim 1.
